(19) United States
(12) Patent Application Publication
(10) Pub. No.: US 2008/0064665 A1
(43) Pub. Date: Mar. 13, 2008

(54) Combination approaches to cancer treatment

(75) Inventors: Adam Vorn Patterson, Auckland (NZ); William Robert Wilson, Waiuku (NZ)

Correspondence Address: NIXON & VANDERHYE, PC, 901 NORTH GLEBE ROAD, 11TH FLOOR, ARLINGTON, VA, 22203, US

(73) Assignee: Auckland Uniservices Limited, Auckland (NZ)

(21) Appl. No.: 11/654,698

(22) Filed: Jan. 18, 2007

(30) Foreign Application Priority Data
Sep. 11, 2006 (NZ) NZ 549831

(51) Int. Cl. A61K 31/66

(52) U.S. Cl. 514/131

(57) ABSTRACT

The present invention relates to a method for the production of an anti-cancer effect in a warm-blooded animal such as a human, which comprises administering to said animal an effective amount of a compound of Formula (I)

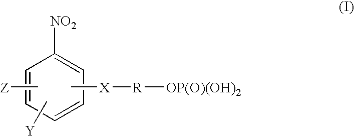

wherein X represents at any available ring position —CONH—, —SO₂NH—, —O—, —CH₂—, NHCO— or —NHSO₂—; R represents a lower C1-6 alkyl optionally substituted with one or more groups including hydroxyl, amino and N-oxides therefrom or dialkylamino and N-oxides therefrom; Y represents at any available ring position —N-aziridinyl, —N(CH₂CH₂W)₂ or —N(CH₂CHMeW)₂, where each W is independently selected from halogen or —OS0₂Me; Z represents at any available ring position —NO₂, -halogen, —CN, —CF₃, or —SO₂Me; or a pharmaceutically acceptable salt or derivative thereof, before, after or simultaneously with an effective amount of docetaxel.

Cancer is a significant cause of death, particularly in industrialised nations. While there are a number of anti-cancer therapies now available, there remains a need for new approaches to treating cancer which offer better outcomes for patients. It is towards one such approach that the present invention is directed.

### SUMMARY OF THE INVENTION

The present invention is broadly based upon the unexpected and surprising finding that compounds of Formula (I) and their salts as defined in WO 2005/042471 used in combination with chemotherapeutic agent docetaxel produces significantly better effects than either agent alone.

Therefore, according to a first aspect of the present invention there is provided a method for the production of an anti-cancer effect in a warm-blooded animal such as a human, which comprises administering to said animal an effective amount of a compound of Formula (I)

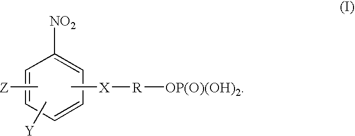

wherein:

### X represents at any available ring position —CONH—, —SO₂NH—, —O—, —CH₂, —NHCO— or —NHSO₂—;

### R represents a lower C1-6 alkyl optionally substituted with one or more groups including hydroxy, amino and N-oxides therefrom or dialkylamino and N-oxides therefrom;

### Y represents at any available ring position —N-aziridinyl, —N(CH₂CH₂W)₂ or —N(CH₂CHMeW)₂, where each W is independently selected from halogen or —OSO₂Me;

### Z represents at any available ring position —NO₂, -halogen, —CN, —CF₃ or —SO₂Me; or a pharmaceutically acceptable salt or derivative thereof, before, after or simultaneously with an effective amount of docetaxel.

Anti-cancer effects include, but are not limited to, anti-tumor effects, the response rate, the time to disease progression and the survival rate. Anti-tumor effects include but are not limited to, inhibition of tumor growth, tumor growth delay, regression of tumor, shrinkage of tumor, increased time to regrowth of tumor on cessation of treatment and slowing of disease progression.

An “effective amount” includes amounts of the compound which provide an anti-cancer effect on their own as well as amounts of the compound which, while being less than a therapeutic dose for the compound as a monotherapy, do provide an anti-cancer effect when the second compound is administered in combination.

According to a further aspect of the present invention there is provided a method for the treatment of a cancer in a warm-blooded animal such as a human, which comprises administering to said animal an effective amount of a compound of Formula (I) as defined above or a pharmaceutically acceptable salt thereof, before, after or simultaneously with an effective amount of docetaxel.

Preferably, in each such method, the compound of Formula (I) or salt thereof and docetaxel may each be administered together with a pharmaceutically acceptable excipient or carrier.

According to a further aspect of the present invention there is provided a therapeutic combination treatment comprising the administration of an effective amount of a compound of Formula (I) as defined above or a pharmaceutically acceptable salt thereof, optionally together with a pharmaceutically acceptable excipient or carrier, and the simultaneous, sequential or separate administration of an effective amount of docetaxel, optionally together with a pharmaceutically acceptable excipient or carrier, to a warm-blooded animal such as a human in need of such therapeutic treatment.

Such therapeutic treatment includes an anti-cancer effect and an anti-tumor effect.

A combination treatment of the present invention as defined herein may be achieved by way of the simultaneous, sequential or separate administration of the individual components of said treatment. A combination treatment as defined herein may be applied as a sole therapy or may involve surgery or radiotherapy or an additional chemotherapeutic agent in addition to a combination treatment of the invention.

Surgery may comprise the step of partial or complete tumor resection, prior to, during or after the administration of the combination treatment described herein.

The effect of a combination treatment of the present invention is expected to be a synergistic effect. According to the present invention a combination treatment is defined as affording a synergistic effect if the effect is therapeutically superior, as measured by, for example, the extent of the response, the response rate, the time to disease progression or the survival period, to that achievable on dosing one or other of the components of the combination treatment at its conventional dose. For example, the effect of the combination treatment is synergistic if the effect is therapeutically superior to the effect achievable with a compound of Formula (I) or docetaxel alone. Further, the effect of the combination treatment is synergistic if a beneficial effect is obtained in a group of patients that does not respond (or responds poorly) to a compound of Formula (I) or docetaxel alone. In addition, the effect of the combination treatment is defined as affording a synergistic effect if one of the components is dosed at its conventional dose and the other component(s) is/are dosed at a reduced dose and the therapeutic effect, as measured by, for example, the extent of the response, the response rate, the time to disease progression or the survival period, is equivalent to that achievable on dosing conventional amounts of the components of the combination treatment. In particular, synergy is deemed to be present if the conventional dose of compound of Formula (I) or docetaxel may be reduced without detriment to one or more of the extent of the response, the response rate, the time to disease progression and survival data, in particular without detriment to the duration of the response, but with fewer and/or less troublesome side effects than those that occur when conventional doses of each component are used.

Combination treatments of the present invention may be used to treat cancer, particularly a cancer involving a solid tumor. In particular such combination treatments of the invention are expected to slow advantageously the growth of primary and recurrent solid tumors of, for example, the ovary, colon, stomach, brain, thyroid, adrenal, pituitary, pancreas, bladder, breast, prostate, lungs, kidney, liver, head and neck (including esophageal), cervix, endometrium, vulva, skin and connective tissues or bone. More especially combination treatments of the present invention are expected to slow advantageously the growth of tumors in colorectal cancer and in lung cancer, for example mesothelioma and non-small cell lung cancer (NSCLC). More particularly such combination treatments of the invention are expected to inhibit any form of cancer associated with VEGF including leukaemia, multiple myeloma and lymphoma and also, for example, to inhibit the growth of those primary and recurrent solid tumors which are associated with VEGF, especially those tumors which are significantly dependent on VEGF for their growth and spread, including for example, certain tumors of the kidney, ovary, colon (including rectum), brain, thyroid, pancreas, bladder, breast, prostate, lung, vulva, skin and particularly NSCLC.

The therapeutic combination of the invention may be administered in the form of a combination product or a pharmaceutical composition. Therefore, according to one further aspect of the present invention there is provided a combination product comprising a compound of Formula (I) as defined above or a pharmaceutically acceptable salt thereof, and docetaxel.

According to a second further aspect of the invention there is provided a pharmaceutical composition which comprises a compound of Formula (I) as defined above or a pharmaceutically acceptable salt thereof, and docetaxel, in association with a pharmaceutically acceptable excipient or carrier.

Kits may also be provided. According to a further aspect of the present invention there is provided a kit comprising a compound of Formula (I) as defined above or a pharmaceutically acceptable salt thereof, and docetaxel.

According to a further aspect of the present invention there is provided a kit comprising:
a) a compound of Formula (I) as defined above or a pharmaceutically acceptable salt thereof in a first unit dosage form;
b) docetaxel in a second unit dosage form; and
c) container means for containing said first and second dosage forms.

### DESCRIPTION OF THE DRAWINGS

FIG. 1: Kaplan-Meier survival plot of H460 xenograft bearing CD-1 nude mice treated with PR-104, docetaxel or a combination of PR-104 and docetaxel on a q2w×2 schedule.

FIG. 2: Kaplan-Meier plot of SiHa xenograft bearing Rag-1 mice treated with PR-104, docetaxel or a combination of PR-104 and docetaxel on a q2w×2 schedule.

FIG. 3: Kaplan-Meier plot of 22RV1 xenograft bearing CD-1 nude mice treated with PR-104, docetaxel or a combination of PR-104 and docetaxel.

FIG. 4: Kaplan-Meier plot of A2780 xenograft bearing mice treated with PR-104, docetaxel or a combination of PR-104 and docetaxel.

FIG. 5: Kaplan Meier survival plot of SiHa xenograft bearing CD-1 nude mice treated with SN 28343 and docetaxel, alone and in combination, on a qw×2 treatment schedule.

FIG. 6: Mean tumor diameter of SiHa xenografts grown in CD-1 nude mice and treated with SN 28343 or docetaxel, alone and in combination, on a qw×2 treatment schedule.

FIG. 7: Activity of docetaxel and SN 28343, alone and in combination, against SiHa tumors in excision assay.

FIG. 8: Activity of docetaxel and SN 29303, alone and in combination, against SiHa tumors in excision assay.

FIG. 9: Schedule dependence of SiHa xenograft cell kill with docetaxel in combination with SN 28343.

FIG. 10: Schedule dependence of SiHa xenograft cell kill with docetaxel in combination with SN 29303.

### DETAILED DESCRIPTION OF THE INVENTION

This invention is primarily based upon the surprising finding of synergism between anti-cancer agents. One agent is the chemotherapeutic agent docetaxel (Taxotere®; chemical name (2R,3S)-N-carboxy-3-phenylisoserine, N-tert-butyl ester, 13-ester with 5β-20-epoxy-1,2α,4,7β,10β,13α-hexahydroxytax-11-en-9-one 4-acetate 2-benzoate, trihydrate); which is commercially available from Aventis Pharmaceuticals. The second agent is a compound of Formula (I) as defined and described in PCT/NZ2004/000275 (published as WO 2005/042471), with the compounds 2-[(2-bromoethyl)-2,4-dinitro-6-[[[2-(phosphonooxy)ethyl]amino]-carbonyl]anilino]ethyl methane sulfonate (known as PR-104), 2-[Bis(2-bromoethyl)amino]-N-(2-hydroxyethyl)-3,5-dinitrobenzamide phosphate ester (known as SN 28343) and 2-[2-bromoethyl)-2,4-dinitro-3-({[3-(phosphonooxy)propyl]amino}carbonyl) anilino]ethyl methanesulfonate (known as SN 29303) being representative.

The agents are administered in combination. It is to be understood that “combination” encompasses the simultaneous or sequential administration of the agents, with “sequential” meaning either agent can be administered before or after the other provided only that the delay in administering the second agent should not be such as to lose the benefit of the combination therapy.

The agents may also be in any appropriate form for administration. Commonly, the agents will be formulated for parenteral injection (including intravenous, subcutaneous, intramuscular, intravascular or infusion) for example as a sterile solution, suspension or emulsion. However, other formulations are in no way excluded.

In general the compositions described herein may be prepared in a conventional manner using conventional excipients and/or carriers, including liposomal or albumin carriers.

Where intended for parenteral injection for example, the component agents can be formulated in accordance with manufacturer's instructions or as described below in the experimental section.

The dosages and schedules of administration of the component agents may be varied according to the particular disease state and overall condition of the patient. Administration may be at single-agent dosages (up to 100 mg/m² for docetaxel) employed in current clinical practice for either agent or for both. More commonly, however, the dose of one or both agents will be reduced below single-agent clinical practice, both to reflect the therapeutic benefit of the combination and to minimise the potential for toxicity. Any and all such dose combinations can be employed subject to the component agents being present in amounts which combine to produce an anti-cancer effect.

The final dose, and dose scheduling, will be determined by the practitioner treating the particular patient using professional skill and knowledge.

A combination treatment of the present invention is most desirably a sole therapy but is not limited to that—it may in addition involve surgery or radiotherapy or the administration of a chemotherapeutic agent.

Surgery may comprise the step of partial or complete tumor resection, prior to, during or after the administration of the combination treatment of the present invention.

Chemotherapeutic agents for optional use with the combination treatment of the present invention may include, for example, the following categories of therapeutic agent:
(i) antiproliferative/antineoplastic drugs and combinations thereof as used in medical oncology (for example carboplatin and cisplatin);
(ii) cytostatic agents, for example inhibitors of growth factor function such as growth factor antibodies, growth factor receptor antibodies (for example the anti-erbB2 antibody trastuzumab and the anti-erbB1 antibody cetuximab), Class I receptor tyrosine kinase inhibitors (for example inhibitors of the epidermal growth factor family), Class II receptor tyrosine kinase inhibitors (for example inhibitors of the insulin growth factor family such as IGF1 receptor inhibitors as described, for example, by Chakravarti et al., Cancer Research, 2002, 62: 200-207), serine/threonine kinase inhibitors, farnesyl transferase inhibitors and platelet-derived growth factor inhibitors;
(iii) antiangiogenic agents such as those which inhibit the effects of vascular endothelial growth factor (for example the anti-vascular endothelial cell growth factor antibody bevacizumab and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; within WO 00/47212), vatalanib (PTK787; WO 98/35985) and SU11248 (WO 01/60814));
(iv) vascular damaging agents such as the compounds disclosed in International Patent Applications WO 99/02166, WO 00/40529, WO 00/41669, WO 01/92224, WO 02/04434 and WO 02/08213;
(v) biological response modifiers (for example interferon); and
(vi) a bisphosphonate such as tiludronic acid, ibandronic acid, incadronic acid, risedronic acid, zoledronic acid, clodronic acid, neridronic acid, pamidronic acid and alendronic acid.

Radiotherapy may be administered according to the known practices in clinical radiotherapy. The dosages of ionising radiation will be those known for use in clinical radiotherapy. The radiation therapy used will include for example the use of y-rays, X-rays, and/or the directed delivery of radiation from radioisotopes. Other forms of DNA damaging factors are also included in the present invention such as microwaves and UV-irradiation. For example X-rays may be dosed in daily doses of 1.8-2.0Gy, 5 days a week for 5-6 weeks.

Normally a total fractionated dose will lie in the range 45-60Gy. Single larger doses, for example 5-10Gy may be administered as part of a course of radiotherapy. Single doses may be administered intraoperatively. Hyperfractionated radiotherapy may be used whereby small doses of X-rays are administered regularly over a period of time, for example 0.1Gy per hour over a number of days. Dosage ranges for radioisotopes vary widely, and depend on the half-life of the isotope, the strength and type of radiation emitted, and on the uptake by cells.

The invention will now be illustrated with reference to the synergistic interaction between docetaxel and representative compounds of Formula (I) in the experimental section which follows.

Experimental

Part 1

Objective

To determine the efficacy of PR-104, docetaxel and schedules thereof against established H460 human lung cancer xenografts.

Materials and Methods

Mice and Husbandry

Specific pathogen-free homozygous nu/nu (CD-1) nu/nu [NIH-III] (Charles River Laboratories, Wilmington, Mass.) were provided by the Animal Resources Unit (University of Auckland) at 7 to 9 weeks of age. Mice were housed in groups of 4-7 in a temperature-controlled room (22±2° C.) with a 12-hour light/dark cycle and were fed ad libitum water and a standard rodent diet (Harlan Teklad diet 2018i). All animals were uniquely identifiable by ear tag number. All animal protocols were approved by the Animal Ethics Committee of the University of Auckland (AEC approval C337).

Xenografts

A single cell suspension was prepared by trypsinisation (1× Trypsin/EDTA) from spinner culture, counted, and suspended in ∝MEM to give required cell concentration, as listed below. Mice were inoculated (100 μL) at a single subcutaneous site (right flank) using a 1 ml syringe with a 26 gauge needle.

| | | | | Cell | Cells/ |
|---|---|---|---|---|---|
| Number | Gender | Strain | Tumor Site | Line | Inoculation |
| 55 | Female | CD-1 nude | Subcutaneous | H460 | 1 × 10⁷ |

Seven days post-inoculation, tumors were measured three times per week until they reached the treatment size (mean diameter 5.8-8.2 mm; average 7.0 mm). Mean tumor diameter was averaged from the longest diameter (length) multiplied by the perpendicular measurement (width). Tumor diameters were estimated when mean diameter was below 5 mm, and measured with electronic callipers when ≧5 mm.

Tumor volume was calculated using the formula:

$$Tumor volume ( {mm}^{3} ) = \frac{π ( L \times {w}^{2} )}{6}$$

where L=length and w=width in mm of the carcinoma.

Test Compounds

PR-104: 2-[(2-Bromoethyl)-2,4-dinitro-6-[[[2-(phosphonooxy)ethyl]amino]-carbonyl anilino]ethyl methanesulfonate.

Docetaxel: Clinical formulation of Taxotere (Aventis Pharma, France). Each vial contains 20 mg docetaxel (0.5 mL of a 40 mg/mL solution) in polysorbate 80. Added solvent is 7 ml of 13% w/w ethanol in water for injection.

PR-104 was dissolved in phosphate buffered saline (PBS), with the addition of one equivalent of sodium bicarbonate (see below). Preparations were briefly vortexed until clear and filter sterilised (0.22 μm). A sample was taken and final concentration was determined by spectrophotometry (using a predetermined extinction coefficient). Typically concentrations of 20-60 mM were prepared. These were held at room temperature in a sterile light-protected glass vial. All solutions were prepared fresh and administered within 4 hours. Excess compound was discarded.

PR-104 was synthesized as the free acid by methods described in WO 2005/042471. Purities were routinely between 92% and 97% as determined by high performance liquid chromatography (HPLC).

Clinical grade Taxotere (manufactured by Aventis) was purchased from A+ Cytotoxic Pharmacy, Auckland Healthcare Services.

Compound Administration Schedule

Test compound administration: doses and schedules

| | | | | | Total | | |
|---|---|---|---|---|---|---|---|
| | | Total Dose | Delay | | Dose | | Female CD- |
| Group | Drug | (mg/kg)^{A} | (hr) | Drug | (mg/kg)^{A} | Schedule | 1 Nude Mice |
| A | Control | | | | | | 8 |
| B | PR-104 | 652 | | | | q2w × 2 | 7 |
| C | Docetaxel | 73 | | | | q2w × 2 | 7 |
| D | PR-104 | 652 | 0 | Docetaxel | 73 | q2w × 2 | 7 |
| E | Docetaxel | 73 | 24 | PR-104 | 652 | q2w × 2 | 7 |
| F | PR-104 | 652 | 24 | Docetaxel | 73 | q2w × 2 | 7 |

^{A}calculated from formula weight of free acids

Growth Delay Experimental Procedure

Tumor bearing mice were assigned randomly to treatment groups when tumor diameter reached treatment size. Animals were rejected if xenografts show evidence of: (i) attachment to underlying muscle (due to risk of local invasion), (ii) signs of ulceration, or (iii) indolent tumor growth. Drug administration begins on the day of assignment. In general, 0.7-0.8 of the inoculated population is assigned to the experiment. Drug administration was undertaken as outlined above.

During and after treatment, tumor size and body weights were measured regularly. Animals were culled if (i) the average diameter of the tumor exceeds 15 mm (survival end-point), (ii) body weight loss exceeds 15% of pre-treatment value, (iii) there is evidence of prolonged or excessive morbidity, or (iv) tumor ulceration occurred. Each experiment was terminated at day 120 after treatment initiation.

Analysis

Efficacy

Kaplan-Meier plots were constructed and median survival was calculated (TTE₅₀). The statistical significance of any differences in overall survival between treatment groups and control was analysed by Log Rank P statistical test. The log-rank test was calculated using XLStat Life (Kovach Computing Services Ltd). The statistic significance in overall survival between each treatment group and control was determined by testing the null hypothesis that the survival curves are identical in the two populations.

The time for individual tumors to increase in volume by 4 fold relative to treatment day-1 (relative tumor volume×4−RTV⁴) was recorded. The median RTV⁴ is calculated for each group and the difference in RTV⁴ between control and treatment groups is described as the Tumor Growth Delay (TGD) in days. RTV⁴ values notmalise for any bias in tumor treatment volume on day-1. The RTV⁴ of each treatment group is tested for statistical difference from control group by unpaired t-test and Mann Whitney U test (means and medians, respectively).

In circumstances where long-term controls (LTCs) occur, an RTV⁴ value equal to the total duration of the experiment is assigned for the purposes of statistical analysis (usually 120 days). Where one or more LTC is present the median RTV⁴ of each treatment group is tested for statistical difference from control group by Mann Whitney U test only. The statistical analysis was conducted at a p level of 0.05 (two-tailed). SigmaStat v3.10 was used for the statistical analysis of RTV⁴ values. SigmaPlot v9 was used for all graph plots.

Toxicity

Weight loss nadirs (time independent maxima) were recorded for each treatment group. Any signs of treatment related morbidity were documented. Acceptable toxicity was defined as no mean group weight loss of over 10% during the test and no individual weight loss over 15%. All unscheduled deaths were recorded.

Results

Summary of growth delay parameters and primary outcomes

| | | | | | | | Weight |
|---|---|---|---|---|---|---|---|
| | | Dose | | | Unscheduled | Day of | loss Nadir |
| Group | Compound | (mg/kg) | Schedule | N | deaths | death | (%) |
| A | Control | | | 8 | 0 | | −1.4 ± 0.8 |
| B | PR-104 | 652 | q2w × 2 | 7 | 0 | | −4.9 ± 1.0 |
| C | Docetaxel | 73 | q2w × 2 | 7 | 0 | | −4.2 ± 1.0 |
| D | PR-104 -0 h- | 652 + 73 | q2w × 2 | 7 | 0 | | −6.2 ± 1.4 |
| | Docetaxel | | | | | | |
| E | Docetaxel | 73 + 652 | q2w × 2 | 7 | 1 | 19^{A} | −8.4 ± 1.6 |
| | 24 hr- PR-104 | | | | | | |
| F | PR-104 - 24 hr- | 652 + 73 | q2w × 2 | 7 | 1 | 20^{B} | −8.8 ± 2.8 |
| | Docetaxel | | | | | | |

^{A}metastasis
^{B}>15% body weight loss

Statistical analysis

| | Overall Survival | | | | Relative Tumor Volume | | | |
|---|---|---|---|---|---|---|---|---|
| | | | Gain | Log | Median | | Unpaired | Mann Whitney |
| | | TTE₅₀ | TTE₅₀ | Rank (P | RTV⁴ | TGD | t-test (P | U test |
| Group | LTC^{A} | (days)^{B} | (days) | value)^{C} | (days)^{D} | (%)^{E} | value)^{F} | (P value)^{F} |
| A | 0 | 11 | — | — | 8 | — | — | — |
| B | 0 | 29 | 18 | <0.001 | 23 | 187.5 | 0.037 | <0.001 |
| C | 0 | 14 | 3 | 0.424 | 8 | 0 | 0.693 | 0.867 |
| D | 0 | 43 | 32 | <0.001 | 37 | 362.5 | <0.001 | <0.001 |
| E | 0 | 41 | 30 | <0.001 | 34.5 | 331.3 | <0.001 | <0.001 |
| F | 0 | 43 | 32 | <0.001 | 35 | 337.5 | <0.001 | <0.001 |

^{A}LTC = long term control (failed to reach end-point within specified duration or experiment)
^{B}TTE₅₀ = median time for tumor end-point to occur from day of treatment
^{C}Log rank test of statistical significance in overall survival probability for each treatment group versus control
^{D}RTV⁴ = relative tumor volume × 4; median time for tumor volume to increase 4-fold from day of treatment
^{E}TGD = tumor growth delay; relative gain in median RTV⁴ versus control (%)
^{F}versus control

Tumor volume on treatment day-1 ranged from 85-281 mm³. Average tumor volume on treatment day-1 was 169±48 mm³ (mean±S.D.).

Controls: The H460 lung cancer xenografts in eight Group A mice receiving no treatment grew progressively, increasing their volume 4-fold (RTV⁴) from day-1 of experimental assignment with a median time of 8 days. The median time for Group A tumors to reached endpoint (>15 mm mean diameter) was calculated as 11 days. All H460 neoplasms grew to endpoint within the 120 day experimental period.

PR-104 treatment: A total dose of 652 mg/kg of PR-104 was administered i.p. (q2w×2), providing a 18-day improvement in median survival that was statistically significant as determined by log rank test (P<0.001). A mean body weight loss nadir of −4.2±1.0% was recorded.

Docetaxel treatment: A total dose of 73 mg/kg of docetaxel was admistered i.p. (q2w×2), providing a 3-day improvement in median survival that was not statistically significant as determined by log rank test (P=0.424). A mean body weight loss nadir of −4.9±1.0% was recorded.

PR-104+Docetaxel treatment: PR-104 (652 mg/kg) −0 hr delay- docetaxel (73 mg/kg) (q2w×2) provided a 29-day tumor growth delay (TDG 363%, P<0.001) which was independently associated with a 32 day increase in median survival, as determined by log tank test (P<0.001). A mean body weight loss nadir of −6.2±1.4% was recorded. No unscheduled deaths were recorded.

Docetaxel (73 mg/kg) −24 hr delay—PR-104 (652 mg/kg) (q2w×2) provided a 26.5-day tumor growth delay (TDG 331%, P<0.001) which was independently associated with an increase in median survival, as determined by log rank test (P<0.001). A mean body weight loss nadir of −8.4±1.6% was recorded. 1 unscheduled death was recorded, due to tumor metastasis.

PR-104 (652 mg/kg) −24 hr delay—docetaxel (73 mg/kg) (q2w×2) provided a 27-day tumor growth delay (TGD 338%, P<0.001) which was independently associated with an increase in median survival, as determined by log rank test (P<0.001). A mean body weight loss nadir of −8.4±1.6% was recorded. 1 unscheduled death was recorded.

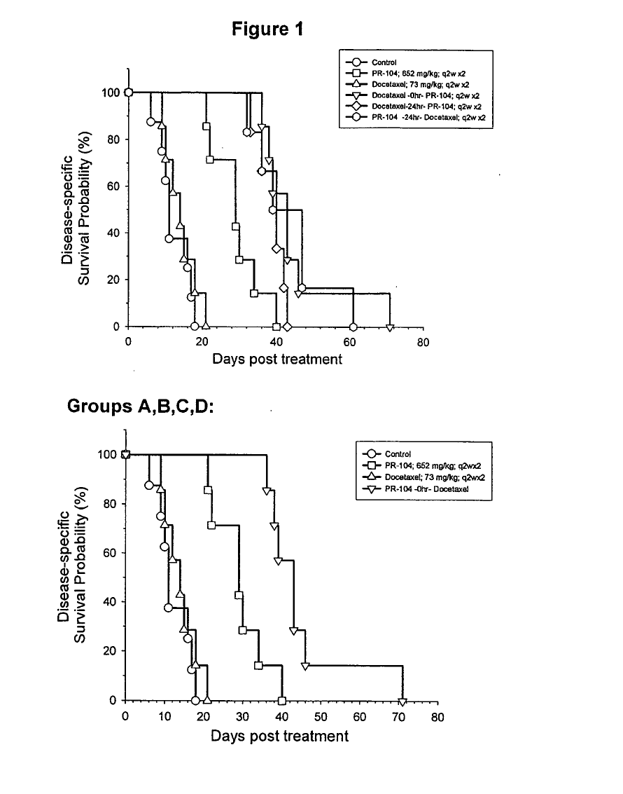

The Kaplan-Meier curves of individual animal survival times are depicted in FIG. 1.

Conclusion

The H460 xenograft is refractory to docetaxel treatment. PR-104 was observed to possess significant single agent activity against the H460 xenograft model as determined by tumor growth delay and survival end-points. The co-administration of PR-104 and docetaxel was active at all schedules. Co-administration of docetaxel+PR-104 resulted in a significant median tumor growth delay (TGD 363%; P<0.001) and was independently associated with an overall survival improvement by log rank test (P<0.001). Delaying the administration of either agent by 24 hr relative to the other was also efficacious but was associated with moderately greater weight loss and 2/14 unscheduled deaths. The combination of PR-104 and docetaxel provided a positive interaction, with both median tumor growth delay and median survival increasing in a manner that was greater than additive. Given the docetaxel resistant nature of the H460 xenograft model, these data indicate that a substantial therapeutic gain has occurred through addition of PR-104 to the docetaxel treatment regimen. Overall there was evidence of a positive intereaction between PR-104 and docetaxel, with both median tumor growth delay and median survival increasing in a manner that was greater than additive.

Part 2

Objective

To determine the docetaxel sensitivity of the SiHa human cervical cancer xenograft in Rag-1^{null} mice, and to evaluate the drug combination of docetaxel+PR-104 against the SiHa xenograft.

Materials and Methods

As for Part 1 except as noted below.

Tumor inoculations

| | | | Tumor | Cell | Cells/ | Injection |
|---|---|---|---|---|---|---|
| Number | Gender | Strain | Site | Line | Inoculation | Volume |
| 50 | female | Rag-1 | Subcu- | SiHa | 8.5 × 10⁶ | 100 μl |
| | | Balb/c | taneous | | | |

Drug administration schedule

| | | Time Delay | | | | | |
|---|---|---|---|---|---|---|---|
| Compound 1 | Dose (mg/kg)^{A} | (hr) | Compound 2 | Dose (mg/kg)^{A} | Schedule | # Mice | |
| Control | | | | | | 9 | |
| PR-104 | 652 | 0 | | | q2w × 2 | 8 | |
| Docetaxel | 73 | 0 | | | q2w × 2 | 9 | |
| PR-104 | 652 | 0 | Docetaxel | 73 | q2w × 2 | 9 | |
| | | | | | | 35 | total |

^{A}Calculated from formula weight of free acids

End-point: After treatment, tumor size and body weights were measured regularly and mice were culled either when the average diameter of the tumor reached 15 mm (end-point), the tumor ulcerated or when the body weight change reached −15%. Experiment was ended and all remaining mice culled 120 days after treatment.

Analysis: End-points will be expressed as TTE_{50,} Median RTV⁴ and plotted in Kaplan-Meier Plots and analysed by Log Rank P statistical test. Weight loss nadir will be compared between schedules.

Results

Summary of treatment parameters

| | | Total Dose | | | Unscheduled | Day of | Weight Loss |
|---|---|---|---|---|---|---|---|
| Group | Compound | (mg/kg) | Schedule | N | deaths | death | Nadir (%) |
| A | PBS | 0 | SD | 9 | 1 | 14^{A-} | −1.1 ± 0.4 |
| B | PR-104 | 652 | q2w × 2 | 8 | 1 | 14^{B} | −2.2 ± 1.2 |
| C | Docetaxel | 73 | q2w × 2 | 9 | 2 | 44^{A}, 66^{C} | −7.0 ± 0.8 |
| D | PR-104 + Docetaxel | 652 + 73 | q2w × 2 | 9 | 1 | 5^{B, D} | −7.0 ± 2.0 |

^{A}Attached tumor
^{B}Tumor metastasis
^{C}Ulceration
^{D}Weight loss >15%

Statistical analysis

| | Overall Survival | | | | Relative Tumor Volume | | | |
|---|---|---|---|---|---|---|---|---|
| | | | Gain | Log | Median | | Unpaired | Mann Whitney |
| | | TTE₅₀ | TTE₅₀ | Rank (P | RTV⁴ | TGD | t-test (P | U test |
| Group | LTC^{A} | (days)^{B} | (days) | value)^{C} | (days)^{D} | (%)^{E} | value)^{F} | (P value)^{F} |
| A | 0 | 17 | — | — | 12 | — | — | — |
| B | 0 | 25 | 8 | 0.044 | 16 | 33.3 | 0.048 | 0.094 |
| C | 0 | 54 | 37 | <0.001 | 44.5 | 270.8 | <0.001 | <0.001 |
| D | 0 | 74 | 57 | <0.001 | 67.5 | 462.5 | <0.001 | <0.001 |

^{A}LTC = long term control (failed to reach end-point within specified duration or experiment)
^{B}TTE₅₀ = median time for tumor end-point to occur from day of treatment
^{C}Log rank test of statistical significance in overall survival probability for each treatment group versus control
^{D}RTV⁴ = relative tumor volume × 4; median time for tumor volume to increase 4-fold from day of treatment
^{E}TGD = tumor growth delay; relative gain in median RTV⁴ versus control (%)
^{F}versus control

Average tumor volume on treatment day-1 was 254±50 mm³ (mean±S.D.).

Controls: The SiHa carcinoma in nine Group A mice receiving phosphate buffered saline (0.02 ml/g) treatment grew progressively, increasing their volume 4-fold (RTV⁴) from day-1 of experimental assignment with a median time of 12 days. The median time for Group A tumors to reach end-point (>15 mm mean diameter) was calculated at 17 days. All SiHa neoplasms grew to end-point within the 120 day experimental period. One animal had to be culled on day 14 post-treatment due to tumor metastasis.

PR-104 treatment: PR-104 at a total dose of 652 mg/kg was administered i.p. (q2w×2), provided a 4-day improvement in tumor growth delay which was not statistically significant but was independently associated with an 8-day increase in median survival that just reached statistical significance as determined by log rank test (P=0.044). A mean body weight loss nadir of −2.2±1.2% was recorded.

Docetaxel treatment: Docetaxel at a total dose of 73 mg/kg administered i.p. (q2w×2), provided a 32.5-day improvement in tumor growth delay (271%, P<0.001) which was independently associated with a 37-day improvement in median survival that was statistically significant as determined by log rank test (P<0.001). A mean body weight loss nadir of −7.0±0.8% was recorded.

PR-104+docetaxel treatment: PR-104 (652 mg/kg)+docetaxel (73 mg/kg) administered i.p. (q2w×2), provided a 55.5-day improvement in tumor growth delay (TGD 462.5%, P<0.001) which was independently associated with a 57-day improvement in median survival that was statistically significant as determined by log rank test (P<0.001). A mean body weight loss nadir of −7.0±2.0% was recorded.

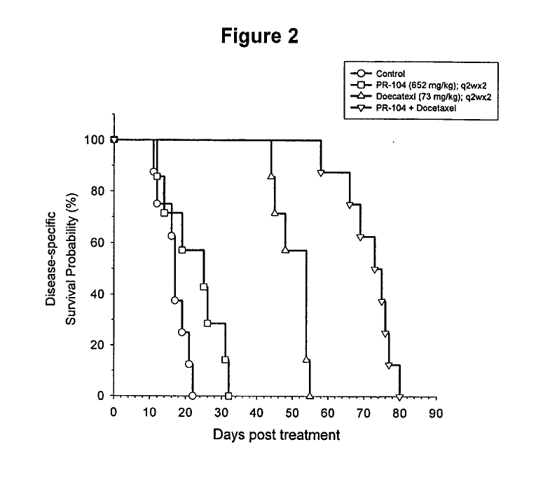

End-points plotted in a Kaplan-Meier graph are shown in FIG. 2.

Conclusion

PR-104 was modestly active as a single agent against the SiHa xenograft model (log rank P=0.044). Docetaxel alone displayed activity, providing a 32.5-day improvement in tumor growth delay (271%, P<0.001) which was independently associated with a 37-day improvement in median survival that was statistically significant as determined by log rank test (P<0.001). In combination PR-104+docetaxel provided a greater than additive 55.5-day improvement in tumor growth delay (TGD 462.5%, P<0.001) which was independently associated with a 57-day improvement in median survival that was statistically significant as determined by log rank test (P<0.001). The maximum body weight loss of the combination treatment was not significantly different from docetaxel administration alone indicating that a large therapeutic gain has occurred. This is an unexpected gain in therapeutic activity and is indicative of a synergistic interaction between these two agents.

Part 3

Objective

To determine the docetaxel sensitivity of the 22RV1 androgen-resistant human prostate cancer xenograft in CD-1 nude mice, and to evaluate the drug combination of docetaxel+PR-104 against 22RV1 xenograft.

### Materials and Methods

As for Part 1 except as noted below.

Tumor inoculations

| | | | Tumor | Cell | Cells/ | Injection |
|---|---|---|---|---|---|---|
| Number | Gender | Strain | Site | Line | Inoculation | Volume |
| 58 | Male | CD-1 | subcu- | 22RV1 | 5 × 10⁶ | 100 μl |
| | | nude | taneous | | | |

Drug administration schedule

| | Dose | Time | Com- | | | | |
|---|---|---|---|---|---|---|---|
| Compound | (mg/ | Delay | pound | Dose | Sched- | | |
| 1 | kg)^{A} | (hr) | 2 | (mg/kg)^{A} | ule | # Mice | |
| Control | — | — | — | — | q2w × 2 | 10 | |
| PR-104 | 652 | — | — | — | q2w × 2 | 9 | |
| Docetaxel | 73 | — | — | — | q2w × 2 | 8 | |
| PR-104 | 652 | 0 | Docetaxel | 73 | q2w × 2 | 9 | |
| | | | | | | 36 | total |

^{A}Calculated from formula weight of free acids

End-point: After treatment, tumor size and body weights were measured regularly and mice were culled either when the average diameter of the tumor reached 15 mm (end-point), the tumor ulcerated or when the body weight change reached −15%. Experiment was ended and all remaining mice culled 120 days after treatment.

Analysis: End-points will be expressed as TTE_{50,} Median RTV⁴ and plotted in Kaplan-Meier Plots and analysed by Log Rank P statistical test.

Results

Summary of treatment toxicity parameters

| | | | | | | | Weight |
|---|---|---|---|---|---|---|---|
| | | Total dose | | | Unscheduled | Day of | loss Nadir |
| Group | Compound | (mg/kg) | Schedule | N | deaths | death | (%) |
| A | PBS | 0 | q2w × 2 | 10 | 0 | | −0.1 ± 0.1 |
| B | Docetaxel | 73 | q2w × 2 | 9 | 0 | | −3.5 ± 0.8 |
| C | PR-104 | 652 | q2w × 2 | 8 | 0 | | −2.1 ± 0.5 |
| D | PR-104 + Docetaxel | 652 + 73 | q2w × 2 | 9 | 1 | 64^{A} | −8.4 ± 1.5 |

^{A}>15% weight loss

Statistical analysis

| | Overall Survival | | | | Relative Tumor Volume | | | |
|---|---|---|---|---|---|---|---|---|
| | | | Gain | Log | Median | | Unpaired | Mann Whitney |
| | | TTE₅₀ | TTE₅₀ | Rank (P | RTV⁴ | TGD | t-test (P | U test |
| Group | LTC^{A} | (days)^{B} | (days) | value)^{C} | (days)^{D} | (%)^{E} | value)^{F} | (P value)^{F} |
| A | 0 | 9.5 | — | — | 9 | — | — | — |
| B | 0 | 24 | 14.5 | <0.001 | 20 | 122 | n/a | 0.014 |
| C | 0 | 26.5 | 17 | <0.001 | 23 | 156 | <0.001 | 0.001 |
| D | 2 | 77.5 | 68 | <0.001 | 72.5 | 706 | <0.001 | 0.002 |

^{A}LTC = long term control (failed to reach end-point within specified duration or experiment)
^{B}TTE₅₀ = median time for tumor end-point to occur from day of treatment
^{C}Log rank test of statistical significance in overall survival probability for each treatment group versus control
^{D}RTV⁴ = relative tumor volume × 4; median time for tumor volume to increase 4-fold from day of treatment
^{E}TGD = tumor growth delay; relative gain in median RTV⁴ versus control (%)
^{F}versus control

Average tumor volume on treatment day-1 was 263±68 mm³ (mean±S.D.). PR-104 treatment: A total dose of 652 mg/kg of PR-104 was administered i.p. (q²w×2), resulted in a median tumor growth delay of 14-days that was significant (TGI=156%, P=0.001). This was independently associated with a 17-day improvement in median survival that was statistically significant as determined by log rank test (P<0.001). A mean body weight loss nadir of −2.1±0.5% was recorded. No deaths occurred.

Docetaxel treatment: A total dose of 73 mg/kg of docetaxel was administered i.p. (q2w×2), provided in a median tumor growth delay of 11-days that was significant (TGI=122%, P<0.014). This was independently associated with a 14.5-day improvement in median survival that was statistically significant as determined by log rank test (P<0.001). A mean body weight loss nadir of −3.5±0.8% was recorded. No deaths occurred.

PR-104+Docetaxel treatment: Co-administration of docetaxel+PR-104 (q2w×2) provided a median tumor growth delay of 62-days that was significant (TGI=689%, P<0.001) which was independently associated with a 68-day improvement in median survival that was statistically significant as determined by log rank test (P<0.001). A mean body weight loss nadir of −8.4±1.5% was recorded. One late death occurred on day 64 of unknown reasons.

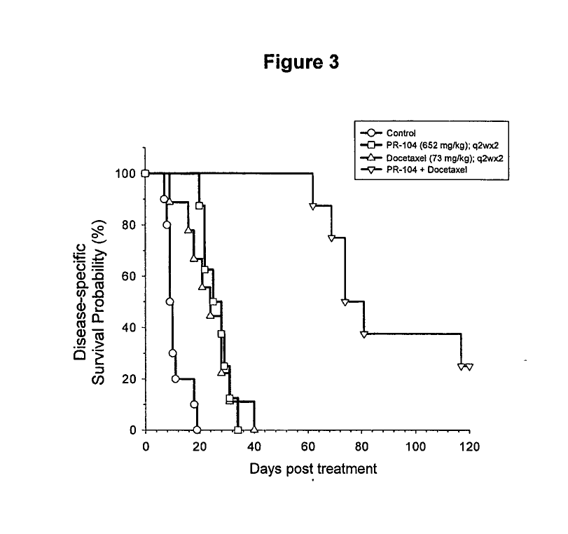

End-points plotted in a Kaplan-Meier graph are shown in FIG. 3.

Conclusion

Docetaxel was moderately active against the 22RV1 prostate xenograft model as a single agent, producing a significant tumor growth delay (TGI=122%, P<0.014). This was associated with increased median survival (14.5-days). PR-104 also had modest single-agent antitumor activity (TGI=TGI=156%, P=0.001), which was related to an improvement in median survival of 17-days (log rank P<0.001). The co-administration of the combination of docetaxel and PR-104 provided a dramatic 62-day improvement in median tumor growth delay (TGI=689%, P<0.001) which was independently associated with a large (68-day) improvement in median survival (log rank test, P<0.001). Co-administration was also associated with 2/9 (22%) complete regressions that failed to regrow by 120-days, indicative of tumor eradication. Thus the combination of these two agents is clearly and unexpectedly synergistic in this model of human prostate cancer.

Part 4

Objective

To determine the efficacy of PR-104, docetaxel and the combination thereof using a q2w×2 schedule against established A2780 human ovarian cancer xenografts growing in CD-1 nude mice.

Materials and Methods

As for Part 1 except as noted below.

Tumor inoculations

| | | | Tumor | Cell | Cells/ | Injection |
|---|---|---|---|---|---|---|
| Number | Gender | Strain | Site | Line | Inoculation | Volume |
| 38 | Female | CD-1 | Sub- | A2780 | 1 × 10⁷ | 100 μl |
| | | nude | cutaneous | | | |

Drug administration schedule

| | | Time | Com- | Dose | | | |
|---|---|---|---|---|---|---|---|
| | Dose | Delay | pound | (mg/ | Sched- | | |
| Compound 1 | (mg/kg)^{A} | (1 hr) | 2 | kg)^{A} | ule | Mice (no.) | |
| Control | | | | | | 8 | |
| PR-104 | 652 | 0 | | | q2w × 2 | 5 | |
| Docetaxel | 73 | 0 | | | q2w × 2 | 5 | |
| PR-104 | 652 | 0 | Docetaxel | 73 | q2w × 2 | 5 | |
| | | | | | | 23 | total |

^{A}Calculated from formula weight of free acid

End-point: After treatment, tumor size and body weights were measured regularly and mice were culled either when the average diameter of the tumor reached 15 mm (end-point), the tumor ulcerated or when the body weight change reached −15%. Experiment was ended and all remaining mice culled 120 days after treatment.

Analysis: End-points will be expressed as TTE_{50,} Median RTV⁴ and plotted in Kaplan-Meier Plots and analysed by Log Rank P statistical test.

Summary of treatment toxicity parameters

| | | Total | | | | | Weight |
|---|---|---|---|---|---|---|---|
| | | Dose | | | Unscheduled | Day of | Loss |
| Group | Compound | (mg/kg) | Schedule | N | Deaths | death | Nadir (%) |
| A | PBS | 0 | q4d × 3 | 8 | 2 | 5^{A}, 27^{B} | −2.6 ± 1.5 |
| B | PR-104 | 652 | q2w × 2 | 5 | 0 | | −6.1 ± 1.2 |
| C | Docetaxel | 73 | q2w × 2 | 5 | 0 | | −8.7 ± 1.5 |
| D | PR-404 + Docetaxel | 652 + 73 | q2w × 2 | 5 | 0 | | −8.5 ± 1.4 |

^{A}Attached tumor
^{B}Found dead

Statistical analysis

| | Overall Survival | | | | Relative Tumor Volume | | | |
|---|---|---|---|---|---|---|---|---|
| | | | Gain | Log | Median | | Unpaired | Mann Whitney |
| | | TTE₅₀ | TTE₅₀ | Rank (P | RTV⁴ | TGD | t-test (P | U test |
| Group | LTC^{A} | (days)^{B} | (days) | value)^{C} | (days)^{D} | (%)^{E} | value)^{F} | (P value)^{F} |
| A | 0 | 6 | — | — | 4.5 | — | — | — |
| B | 0 | 10 | 4 | 0.533 | 8 | 77.8 | n/a | 0.009 |
| C | 0 | 14 | 8 | 0.228 | 12 | 166.7 | n/a | 0.004 |
| D | 1 | 32 | 26 | 0.015 | 27 | 500.0 | n/a | 0.010 |

^{A}LTC = long term control (failed to reach end-point within specified duration or experiment)
^{B}TTE₅₀ = median time for tumor end-point to occur from day of treatment
^{C}Log rank test of statistical significance in overall survival probability for each treatment group versus control
^{D}RTV⁴ = relative tumor volume × 4; median time for tumor volume to increase 4-fold from day of treatment
^{E}TGD = tumor growth delay; relative gain in median RTV⁴ versus control (%)
^{F}versus control

Average tumor volume on treatment day-1 was 226±65 mm³ (mean±S.D.).

Controls: The A2780 carcinomas in eight group A mice receiving phosphate buffered saline (0.02 ml/g) treatment grew progressively, increasing their volume 4-fold (RTV) from day-1 of experimental assignment with a median time of 4.5 days. The median time for Group A tumors to reach end-point (>15 mm mean diameter) was calculated as 6 days. All A2780 neoplasms grew to end-point within the 120-day experimental period. The tumor burden was associated with some weight loss (−2.6±1.5%). One animal was found to have severe body dehydration and reduced mobility on day 5 post-treatment. Necropsy showed tumor invasion into the small intestine. A second animal was found dead on day 27 post-treatment. Necropsy identified no abnormalities.

PR-104 treatment: A total dose of 652 mg/kg of PR-104 was administered i.p. (q2w×2), providing a small but significant 3.5-day improvement in median tumor growth delay (TGD 78%, P=0.009), which was independently associated with a 4-day improvement in median survival that was not statistically significant (P=0.533). A mean body weight loss nadir of −6.1±1.2% was recorded. No unscheduled deaths occurred.

Docetaxel treatment: A total dose of 73 mg/kg of docetaxel was administered i.p. (q2w×2), providing a significant 7.5-day improvement in median tumor growth delay (TGD 167%, P=0.004), which was independently associated with an 8-day improvement in median survival that failed to reach statistical significance (P=0.228). A mean body weight loss nadir of −8.7±1.5% was recorded. No unscheduled deaths occurred.

Combination of PR-104 and Docetaxel treatment: The combination of 652 mg/kg PR-104+73 mg/kg docetaxel (q2w×2) provided a significant 22.5-day improvement in median tumor growth delay (TGD 500%, P=0.01), which was independently associated with a 26-day improvement in median survival that was significant as determined by log rank test (P=0.015). A mean body weight loss nadir of −8.5±1.4% was recorded. No unscheduled deaths occurred.

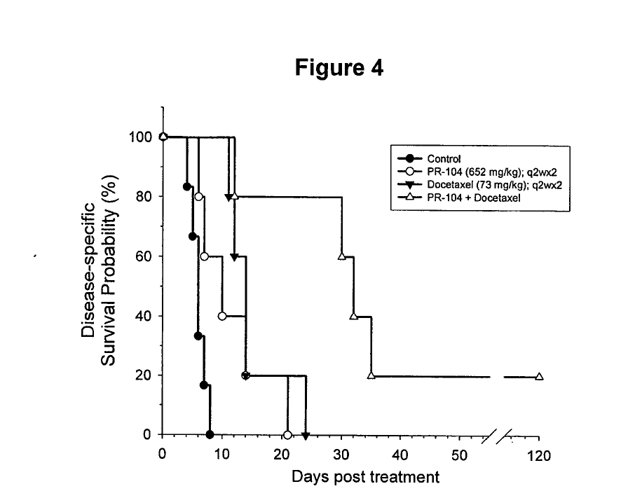

End-points are plotted on a Kaplan-Meier graph as shown in FIG. 4.

Conclusion

PR-104 (at 652 mg/kg) showed no activity based on tumor growth delay. Docetaxel (73 mg/kg) showed a modest but significant 7.5-day tumor growth delay. However, the combination of PR-104 and docetaxel was highly active and produced a large growth delay (TGD 500%) that was substantially greater than additive. In addition, neither agent alone provided a significant survival advantage whereas the combination of PR-104 and docetaxel provided a large gain in therapeutic activity indicative of a synergistic interaction between these two agents against the A2780 xenograft.

Part 5

Objective

To determine the efficacy of SN 28343 and docetaxel, alone and in combination, against established SiHa cervical cancer xenografts.

Materials and Methods

As for Part 1 except as noted below.

| Gender | Strain | Tumor Site | Cell Line | Cells/Inoculation |
|---|---|---|---|---|
| Female | CD-1 nude | Subcutaneous | SiHa | 1 × 10⁷ |

Test Compounds:

SN 28343: 2-[Bis(2-bromoethyl)amino]-N-(2-hydroxyethyl)-3,5-dinitrobenzamide phosphate ester.

Docetaxel: Clinical formulation of Taxotere (Aventis Pharma, France). Each vial contains 20 mg docetaxel (0.5 mL of a 40 mg/mL solution) in polysorbate 80. Added solvent is 7 ml of 13% w/w ethanol in water for injection.

SN 28343 was synthesized as the monosodium salt by the method described in WO 2005/042471. Purity was determined as 93% by HPLC.

SN 28343 was dissolved in phosphate buffered saline (PBS) or saline (see below) with the addition of one equivalent of sodium bicarbonate (see below). Preparations were briefly vortexed until clear and filter sterilised (0.22 μm). A sample was taken and final concentration was determined by spectrophotometry (using a predetermined extinction coefficient). Typically concentrations of 20-60 mM were prepared. These were held at room temperature in a sterile light-protected glass vial. All solutions were prepared fresh and administered within 4 hours. Excess compound was discarded.

Clinical grade docetaxel (Taxotere™; Aventis) was purchased from A+ Cytotoxic Pharmacy, Auckland Healthcare Services. Vials containing 20mg docetaxel in polysorbate 80 (0.5 mL) were diluted with supplied diluent (13% (w/w) ethanol in water).

Compound Administration Schedule

Test compound administration: doses and schedules

| | | | | | | | | | Female |
|---|---|---|---|---|---|---|---|---|---|
| | | Total | | Time | | Total | | | CD-1 |
| | | Dose | | delay | | Dose | | Injection | Nude |
| Group | Compound 1 | (mg/kg)^{A} | | (hr) | Compound 2 | (mg/kg)^{A} | Schedule | Route | Mice |
| A | Saline | 0.015 | ml/g | — | — | — | qw × 2 | i.p. | 6 |
| G | SN 28343 | 513 | | — | — | — | qw × 2 | i.p. | 7 |
| H | Docetaxel | 65 | | — | — | — | qw × 2 | i.p. | 7 |
| J | Docetaxel | 65 | | 0 hr | SN 28343 | 513 | qw × 2 | i.p. | 7 |

^{A}calculated from formula weight of free acids

Results

Summary of experimental parameters and primary outcomes

| | | | | | | | Weight |
|---|---|---|---|---|---|---|---|
| | | Dose | | | Unscheduled | Day of | loss Nadir |
| Group | Compound | (mg/kg) | Schedule | N | deaths | death | (%) |
| A | Saline | | | 6 | — | — | −0.2 ± 0.9 |
| G | SN 28343 | 513 | qw × 2 | 7 | 1 | 104^{I} | −1.6 ± 0.8 |
| H | Docetaxel | 65 | qw × 2 | 7 | 0 | | −4.1 ± 0.8 |
| J | Docetaxel + SN 28343 | 65 + 513 | qw × 2 | 7 | 2^{W,H} | 86^{W} 90^{H} | −6.7 ± 1.3 |

^{I}Infected eye
^{W}>15% weight loss
^{H}Possible internal haemorrhage

Statistical analysis

| | | Overall Survival | | | Relative Tumor Volume | | | |
|---|---|---|---|---|---|---|---|---|
| | | | Gain | | Median | | Unpaired t- | Mann |
| | | TTE₅₀ | TTE₅₀ | Log Rank | RTV⁴ | | test (P | Whitney test |
| Group | LTC^{A} | (days)^{B} | (days) | P value^{C} | (days)^{D} | % TGD^{E} | value)^{F} | (P value)^{F} |
| A | 0 | 21 | — | — | 15 | — | — | — |
| G | 1 | 34 | 13 | P < 0.001 | 30 | 100 | n/a | P = 0.030 |
| H | 2 | 57 | 36 | P < 0.001 | 48 | 220 | n/a | P = 0.004 |
| J | 3 | 81 | 60 | P < 0.001 | 77.5 | 417 | P < 0.001 | P = 0.007 |

^{A}LTC = long term control (failed to reach end-point within specified duration of experiment)
^{B}TTE₅₀ = median time for tumor end-point to occur from day of treatment
^{C}Log rank test of statistical significance in overall survival probability between each treatment group and control
^{D}RTV⁴ = Relative Tumor Volume × 4; median time for tumor volume to increase four-fold from day of treatment
^{E}TGD = Tumor Growth Delay; Relative gain in median RTV⁴ versus control (%)
^{F}versus control

Average tumor volume on treatment day-1 was 294±67 mm³ (mean±SD).

Controls: The SiHa carcinomas in six Group A mice receiving saline treatment grew progressively, increasing their volume 4-fold (RTV⁴) from day-1 of experimental assignment with a median time of 15-days. The median time for Group A tumors to reached endpoint (>15 mm mean diameter) was calculated as 21-days. All SiHa tumors grew to endpoint within the 120 day experimental period.

SN 28343 treatment: A total dose of 513 mg/kg was administered (i.p.; qw×2) which provided a 15-day increase in tumor growth delay which was statistically significant (TGD 100%, P=0.030), and was independently associated with a significant 13-day improvement in median survival that was as determined by log rank test (P<0.001). A mean body weight loss nadir of −1.6±0.8% was recorded. I unscheduled death occurred on day 104 post treatment due to an eye infection.

Docetaxel treatment: A total dose of 65 mg/kg administered i.p. (qw×2), provided a 33-day improvement in tumor growth delay (TGD 220%, P=0.004) and a 36-day increase in median survival that was statistically significant as determined by log rank test (P<0.001). A mean body weight loss nadir of −4.1±0.8% was recorded. No unscheduled deaths occurred.

Docetaxel+SN 28343 treatment: Docetaxel −0 hr delay—SN 28343 administered i.p. (qw×2) provided a 62.5-day tumor growth delay (TDG 417%, P=0.007) which was independently associated with a 60-day increase in median survival, as determined by log rank test (P<0.001). A mean body weight loss nadir of −6.7±1.3% was recorded. 2 unscheduled deaths were recorded late in the study, one due to weight loss >15% (Day-86 post treatment) and the second due to weight loss and apparent internal haemorrhaging (Day-90 post treatment).

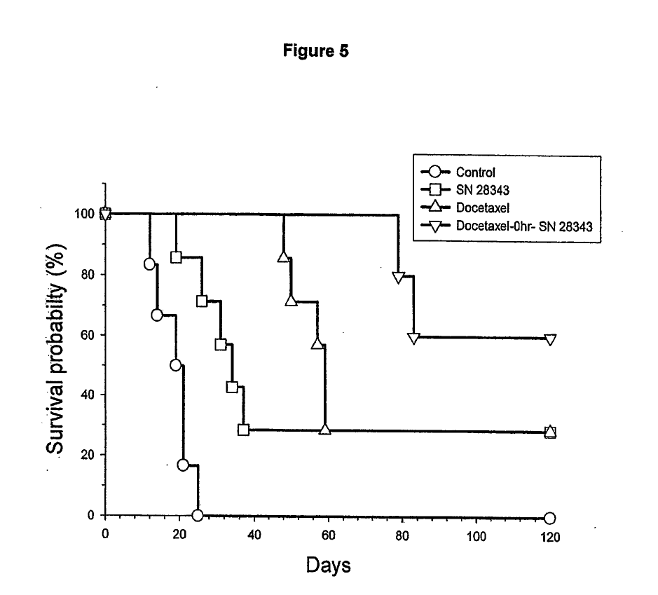

The Kaplan-Meier curves of individual animal survival times are depicted in FIG. 5.

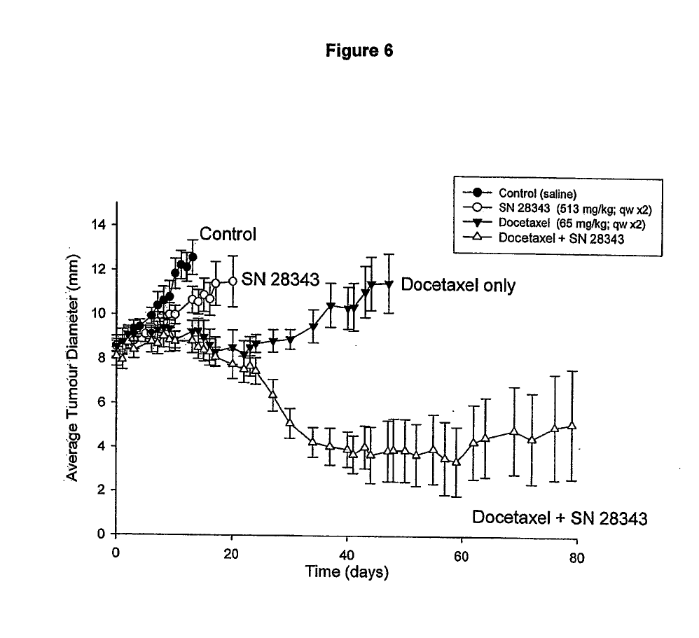

Tumor growth curves are depicted in FIG. 6.

Conclusion

Employing a qw×2 schedule, SN 28343 was observed to possess significant single agent activity against the SiHa xenograft model as determined by tumor growth delay and survival endpoints. Docetaxel was also found to be active against SiHa as a single agent. The co-administration of SN 28343 and docetaxel was active at this dosing schedule. Co-administration of docetaxel+SN 28343 resulted in a significant median tumor growth delay (TGD 417%; P=0.007) and was independently associated with an overall survival improvement by log rank test (P<0.001). The combination of SN 28343 and docetaxel provided a supra-additive interaction, with both median tumor growth delay and median survival increasing in a manner that was greater than expected.

Part 6

Objective

To determine the activity of SN 28343 and SN 29303 alone and in combination with docetaxel at three dosing schedules against established SiHa cervical cancer xenografts in an excision assay in CD-1 Fox^{nu} mice.

Materials and Methods

As for Part 1 except for except where noted below.

Inoculation sets for excision assays

| EXA | | | | | Cell | Cells/ |
|---|---|---|---|---|---|---|
| code | Number | Gender | Strain | Tumor site | line | inoculation |
| SH7a | | | CD-1 | Subcutaneous | SiHa | 1 × 10⁷ |
| | | | Fox^{nu} | | | |

Test Compounds and Their Formulation

SN 28343: 2-[Bis(2-bromoethyl)amino]-N-(2-hydroxyethyl)-3,5-dinitrobenzamide phosphate ester.

SN 29303: 2-[(2-bromoethyl)-2,4-dinitro-3-({[3-(phosphooxy)propyl]amino}carbonyl)anilino]ethyl methanesulfonate.

Docetaxel: Clinical formulation of Taxotere (Aventis Pharma, France). Each vial contains 20 mg docetaxel (0.5 mL of a 40 mg/mL solution) in polysorbate 80. Added solvent is 7 ml of 13% w/w ethanol in water for injection.

SN 28343 was synthesized as the monosodium salt by the methods described in WO 2005/042471. Purity was determined as 93% by HPLC. SN 29303 was synthesized as the free add also by the methods described in WO 2005/042471. Purity was determined as 95% by HPLC.

SN 28343 and SN 29303 were dissolved in phosphate buffered saline (PBS) or saline (see below), with the addition of one equivalent of sodium bicarbonate (see below). Preparations were briefly vortexed until clear and filter sterilised (0.22 μm Ministart disposable filter, Sartorius®). A sample was taken and final concentration was determined by spectrophotometry (using a predetermined extinction coefficient). Typically concentrations of 20-60 mM were prepared. These were held at room temperature in a sterile light-protected glass vial. All solutions were prepared fresh and administered within 4 hours. Excess compound was discarded.

Clinical grade docetaxel (Taxotere™; Aventis) was purchased from A+ Cytotoxic Pharmacy, Auckland Healthcare Services. Vials containing 20 mg docetaxel in polysorbate 80 (0.5 mL) were diluted with supplied diluent (13% (w/w) ethanol in water).

Treatment

Mice with tumors of mean weight 476 mg ±136 (mean±s.d.) were randomly assigned to groups for treatment. Date, body weights (used to adjust injection volume), tumor diameter, unique identifier (tail markings), body weight, and volume to be injected were recorded. Animals were dosed with the test articles i.p. following a defined treatment schedule:

Compound administration schedule

| | | | Dose 1 | | Delay | | | Dose 2 |
|---|---|---|---|---|---|---|---|---|
| Group | n | Treatment 1 | (mg/kg)^{A} | Route 1 | (hr) | Treatment 2 | Route 2 | (mg/kg)^{A} |
| A | 4 | Control | | | | | | |
| B | 4 | Docetaxel | 65 | i.p. | — | — | — | — |
| C | 4 | SN 28343 | 91.2 | i.p. | — | — | — | — |
| D | 3 | SN 29303 | 375 | i.p. | — | — | — | — |
| E | 4 | Docetaxel | 65 | i.p. | 0 | SN 28343 | i.p. | 91.2 |
| F | 4 | Docetaxel | 65 | i.p. | 0 | SN 29303 | i.p. | 375 |
| G | 4 | Docetaxel | 65 | i.p. | 2 | SN 28343 | i.p. | 91.2 |
| H | 4 | Docetaxel | 65 | i.p. | 2 | SN 29303 | i.p. | 375 |
| I | 4 | SN 28343 | 91.2 | i.p. | 2 | Docetaxel | i.p. | 65 |
| J | 4 | SN 29303 | 375 | i.p. | 2 | Docetaxel | i.p. | 65 |

Time delay in co-ordination of two agents was less than 15 minutes
^{A}calculated from formula weight of free acids

Excision Assay

18 hours after treatment the mice were culled by cervical dislocation and tumors removed by dissection, in a sterile laminar flow hood. Whole tumor weights were recorded. Tumors were minced using scissors or scalpels until a fine minceate was obtained, and up to 500 mg of minceate was transferred into a pre-tiered Falcon®14 ml test tube containing a sterile magnetic spin bar and re-weighed.

Chilled, filter-sterilised enzyme cocktail (Pronase (Sigma P-5147, 2.5 mg/ml), Collagenase (Sigma C-5138, 1 mg/ml) and DNAase I (Sigma DN-25, 0.2 mg/ml) in culture medium (αMEM+10% FCS+PS) at 1 ml/50 mg tumor was added and held on ice until all samples were ready (up to 1.5 hr).

Samples were then incubated in 37° C. water bath for 30 min over a magnetic stirrer.

After incubation any undissociated material was allowed to settle for 1 minute. 1 ml of digest was added to 9 ml of medium and spun (Jouan GR 4.11, 1000 rpm) for 8 min. Pellets were re-suspended in 10 ml of medium. Cells/ml was determined using an electronic particle counter (Beckman Coulter Electronics, Z2 model). Samples were then diluted to 1×10⁵cells/ml and 6-fold serial dilutions made down to 4.6×10²cells/ml.

1 ml of each sample was plated in triplicate for each dilution into appropriately labelled Falcon® P-60 tissue culture dishes containing 4 ml of αMEM+10% FCS+PS.

Plates were incubated in 5% CO₂ incubators at 37° C. for 14 days then stained with 1% methylene blue in 50% EtOH.

Where possible, all plates were counted; those colonies counted=larger than 50 cells, confirmed with the light microscope. TMTC was recorded for those plates where there were too many colonies to count.

Data Analysis

The criteria for selecting the best dilution to use for calculating plating efficiency (PE):

Higher dilution count>100 colonies (average); lower dilution 10-100 colonies, use PE from the dilution with fewer colonies.

Higher dilution count >100; lower dilution count <20, use average data from both.

Higher dilution count <100; lower dilution count <20, use PE from the dilution with more colonies.

Higher dilution count <100; lower dilution count >20, use average data from both. Note: For the 10⁵ dilution, take as the “lower limit” colony count a total count of 30, (sum of all replicates).

Statistical analysis was conducted at an overall significance level of 0.05 using one way ANOVA with Holm-Sidak test (SigmaStat v3.5) to complete pairwise multiple comparison procedures for the SN 28343 and SN 29303 groups separately.

Results

Log cell kill versus controls (Mean ± SEM)

| | Treatment group | | | | | | | | |
|---|---|---|---|---|---|---|---|---|---|
| | | | | Doc-0 hr- | Doc-2 hr- | SN 28343 | Doc-0 hr- | Doc-2 hr- | SN 29303- |
| | Docetaxel | SN 28343 | SN 29303 | SN 28343 | SN 28343 | 2 hr-Doc | SN 29303 | SN 29303 | 2 hr-Doc |
| Log Cell Kill | 0.451 ± | 0.821 ± | 0.770 ± | 1.948 ± | 2.127 ± | 1.854 ± | 2.519 ± 0.109 | 2.955 ± 0.155 | 2.215 ± 0.136 |
| (Mean ± SEM) | 0.123 | 0.228 | 0.179 | 0.128 | 0.141 | 0.102 | | | |
| N | 4 | 3 | 4 | 4 | 4 | 4 | 4 | 4 | 4 |

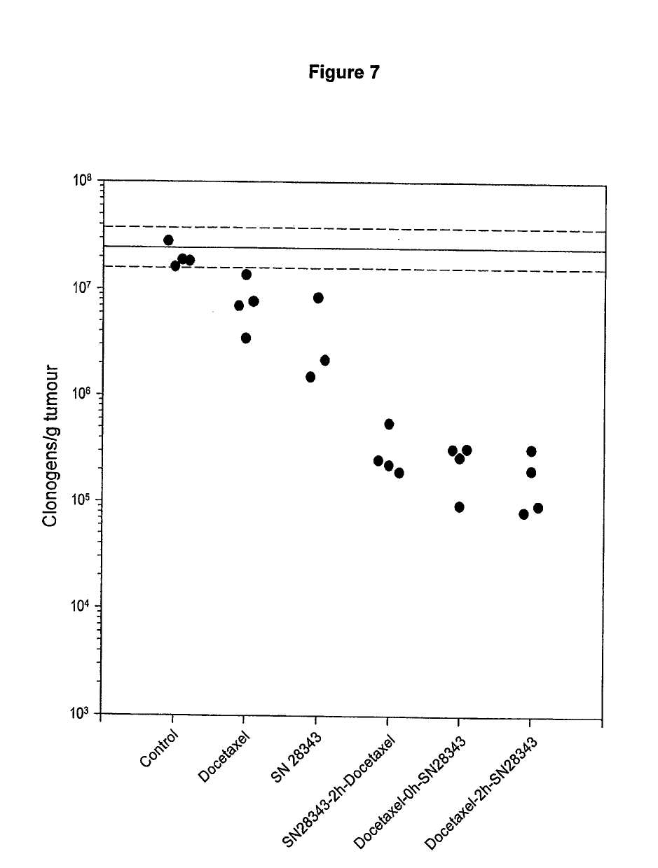

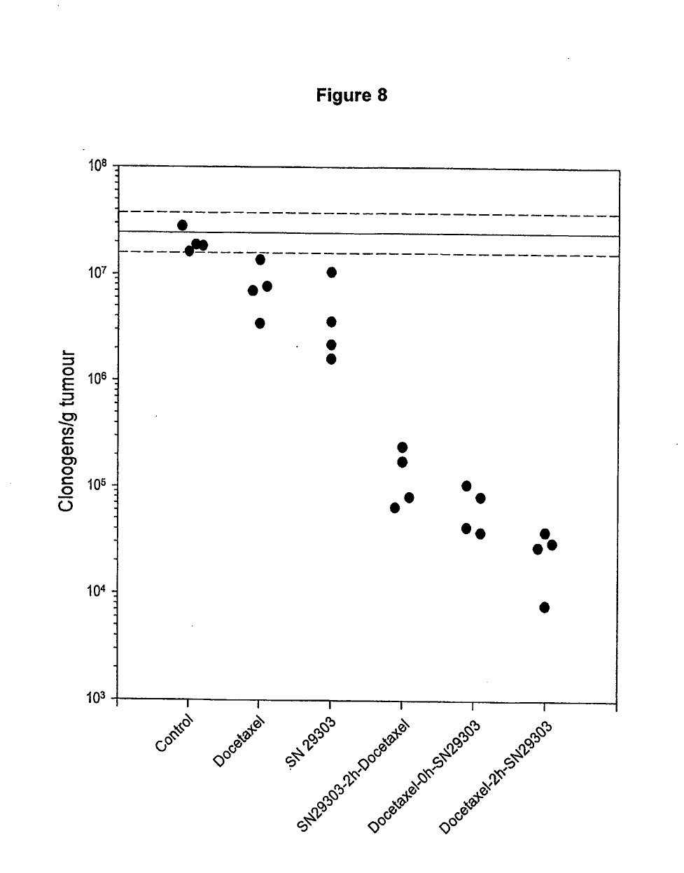

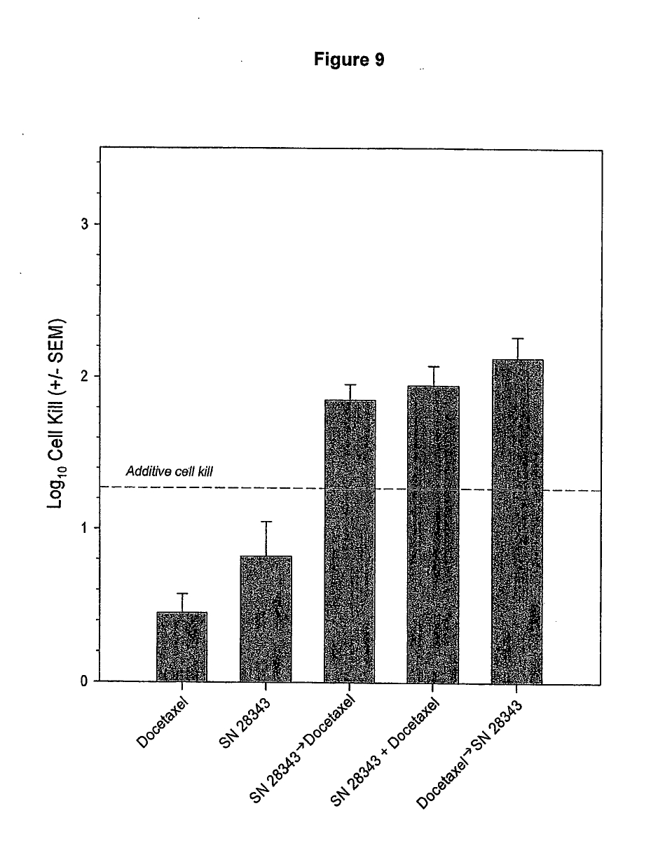

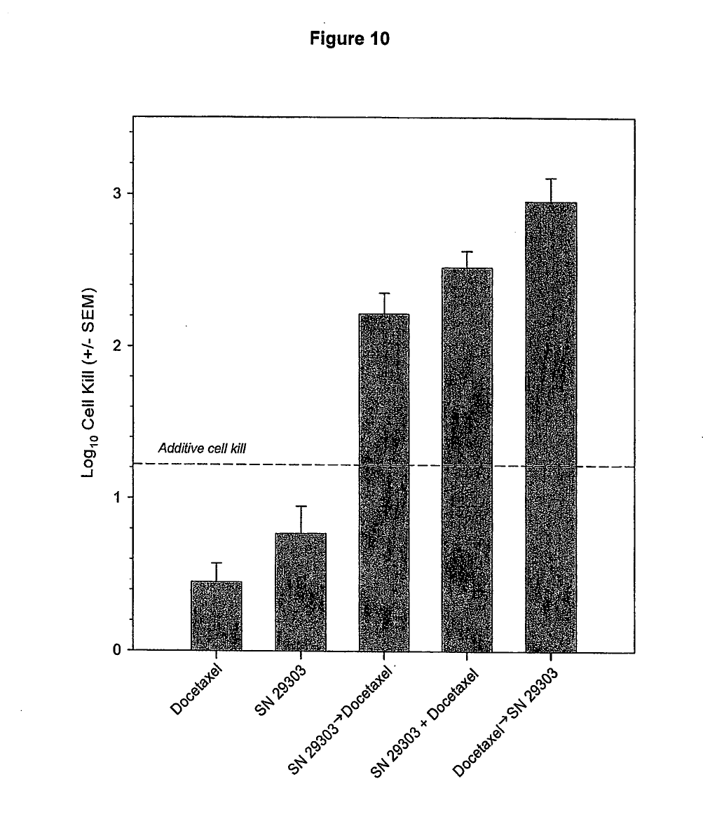

These results are shown in FIGS. 7 to 10.

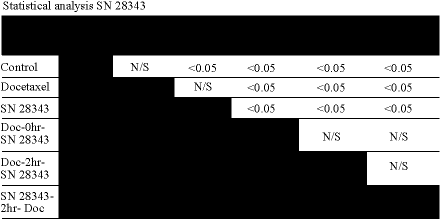

One way ANOVA, All Pairwise Multiple comparison procedures (Holm-Sidak method). Overall significance level=0.05; N/S=not significant.

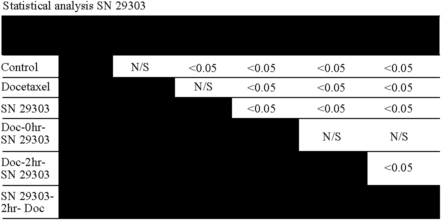

One way ANOVA, All Pairwise Multiple comparison procedures (Holm-Sidak method). Overall significance level 0.05; N/S=not significant.

Conclusions

Docetaxel alone (65 mg/kg; ip.) was inactive against the SiHa human cervical tumor xenograft, failing to produce statistically significant cell kill versus controls. SN 28343 and SN 29303 each provided moderate and significant cell killing as single agents. The combination of docetaxel with either SN 28343 or SN 29303 resulted in more tumor cell killing than would be expected from the independent effects of the two drugs upon co-administration. The positive interaction was achieved irrespective of the dosing regimen used. For SN 28343 no significant difference between any schedule was found. However for SN 29303, when dosing was delayed for 2 hours following docetaxel administration, greater cell killing was obtained in comparison with the reciprocal schedule i.e. SN 29303-2 hr-docetaxel group. This observation was significant by post hoc multiple comparison procedure (p=0.000884, Holm-Sidak test).

Overall there was evidence of a positive interaction between SN 28343 and docetaxel, and between SN 29303 and docetaxel. The interaction was markedly greater than was expected indicating that 3,5-dinitrobenzamide-6-mustard phosphate (Class B) and 2,4-dinitrobenzamide-1-mustard phosphate (Class D) prodrugs of Formula (I) with distinct regio-isomer patterns and with different mustard leaving group arrangements can synergise with docetaxel in vivo.

### INDUSTRIAL APPLICATION

The present invention provides a new approach to cancer therapy. The approach involves administration of two agents in combination to generate anti-cancer effects, including anti-tumor effects. These effects are synergistic.

The agents concerned are docetaxel and a compound of Formula (I) as described in WO 2005/042471. The results for representative compounds of Formula (I) are included in the experimental section to illustrate the general synergism which exists between docetaxel and the various classes of mustard compounds coveted by the wider formula. However, those results, and the representative compounds selected, are in no way a limitation of the invention. Compounds of Formula (I) other than those exemplified can also be selected for combination with docetaxel.

Similarly, the dosages and scheduling exemplified should not be regarded as limiting, with all variations to produce the best therapeutic effect for a particular patent being a matter of selection by the responsible practitioner. That selection may include a specific sequence of administration of docetaxel and the compound of Formula (I) as in the case of SN 29303, for example, to secure maximum patient benefit.

### SUMMARY

The results given above clearly demonstrate a synergistic interaction between representative compounds of Formula (I) and docetaxel across a range of xenograft models indicative of broad application of the combination in cancers as diverse as prostate, cervical, lung and ovarian. The combination represents a significant advance over single agent treatment.

While the present invention is broadly as described above, those persons skilled in the art will appreciate that the specific description is illustrative only and that variations may be made without departing from the invention. For example, combinations of docetaxel with compounds of Formula (I) other than PR-104, SN 28343 and SN 29303 are contemplated, as are variations in the dosing regimens specifically described.

All publications referenced above are incorporated herein in their entirety.

1. A method for the production of an anti-cancer effect in a warm-blooded animal such as a human, which comprises administering to said animal an effective amount of a compound of Formula (I) 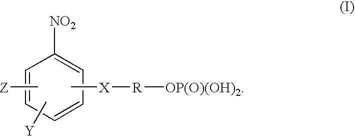
wherein:
X represents at any available ring position —CONH—, —SO₂NH—, —O—, —CH₂—, —NHCO— or —NHSO₂—;
R represents a lower C1-6 alkyl optionally substituted with one or more groups including hydroxyl, amino and N-oxides therefrom or dialkylamino and N-oxides therefrom;
Y represents at any available ring position —N-aziridinyl, —N(CH₂CH₂W)₂ or —N(CH₂CHMeW)₂, where each W is independently selected from halogen or —OSO₂Me;
Z represents at any available ring position —NO₂, -halogen, —CN, —CF₃ or —SO₂Me; or a pharmaceutically acceptable salt or derivative thereof, before, after or simultaneously with an effective amount of docetaxel.

2. The method of claim 1 in which both the compound of Formula (I) or salt thereof and docetaxel are administered together with a pharmaceutically acceptable excipient or carrier.

3. The method of claim 1 in which the compound of Formula (I) is 2[(2-bromoethyl)-2,4-dinitro-6-[[[2-(phosphonooxy)ethyl]amino]-carbonyl]amino]ethyl methanesulfonate.

4. The method of claim 1 in which the compound of Formula (I) is selected from 2-[Bis(2-bromoethyl)amino]-N-(2-hydroxyethyl)-3,5-dinitrobenzamide phosphate ester and 2-[2-bromoethy)-2,4-dinitro-3-({[3-(phosphonooxy)propyl]amino}carbonyl)anilino]ethyl methanesulfonate.

5. A method for the treatment of a cancer in a warm-blooded animal such as a human, which comprises administering to said animal an effective amount of a compound of Formula (I) as defined in claim 1 or a pharmaceutically acceptable salt thereof, before, after or simultaneously with an effective amount of docetaxel.

6. The method of claim 5 in which both the compound of Formula (I) or salt thereof and docetaxel are administered together with a pharmaceutically acceptable excipient or carrier.

7. The method of claim 5 in which the compound of Formula (I) is 2[(2-bromoethyl)-2,4-dinitro-6-[[[2-(phosphonooxy)ethyl]amino]-carbonyl]anilino]ethyl methanesulfonate.

8. The method of claim 5 in which the compound of Formula (I) is selected from 2-Bis(2-bromoethyl)amino]-N-(2-hydroxyethyl)-3,5-dinitrobenzamide phosphate ester and 2-[2-bromoethyl)-2,4-dinitro-3-({[3-(phosphonooxy)propyl]amino}carbonyl)anilino]ethyl methanesulfonate.

9. A therapeutic combination treatment comprising the administration of an effective amount of a compound of Formula (I) as defined in claim 1 or a pharmaceutically acceptable salt thereof, optionally together with a pharmaceutically acceptable excipient or carrier, and the simultaneous, sequential or separate administration of an effective amount of docetaxel, optionally together with a pharmaceutically acceptable excipient or carrier, to a warm-blooded animal such as a human in need of such therapeutic treatment.

10. The treatment of claim 9 in which the compound of Formula (I) is 2[(2-bromoethyl)-2,4-dinitro-6-[[[2-(phosphonooxy)ethyl]amino]-carbonyl]anilino]ethyl methanesulfonate.

11. The treatment of claim 9 in which the compound of Formula (I) is selected from 2-[Bis(2-bromoethyl)amino]-N-(2-hydroxyethyl)-3,5-dinitrobenzamide phosphate ester and 2-[2-bromoethyl)-2,4-dinitro-3-({[3-(phosphonooxy)propyl]amino}carbonyl)anilino]ethyl methanesulfonate.

12. A combination product comprising a compound of Formula (I) as defined in claim 1 or a pharmaceutically acceptable salt thereof, and docetaxel, for use in a method of treatment of a human or animal body by therapy.

13. The product of claim 12 in which the compound of Formula (I)is 2[(2-bromoethyl)-2,4-dinitro-6-[[[2-(phosphonooxy)ethyl]amino]-carbonyl]anilino]ethyl methanesulfonate.

14. The product of claim 12 in which the compound of Formula (I)is selected from 2-[Bis(2-bromoethyl)amino]-N-(2-hydroxyethyl)-3,5-dinitrobenzamide phosphate ester and 2-[2-bromoethyl)-2,4-dinitro-3-({[3-(phosphonooxy)propyl]amino}carbonyl)anilino]ethyl methanesulfonate.

15. A pharmaceutical composition which comprises a compound of Formula (I) as defined in claim 1 or a pharmaceutically acceptable salt thereof, and docetaxel, in association with a pharmaceutically acceptable excipient or carrier.

16. The composition of claim 15 in which the compound of Formula (I) is 2[(2-bromoethyl)-2,4-dinitro-6-[[[2-(phosphonooxy)ethyl]amino]-carbonyl]anilino]ethyl methanesulfonate.

17. The composition of claim 15 in which the compound of Formula (I) is selected from 2-[Bis(2-bromoethyl)amino]-N-(2-hydroxyethyl)-3,5-dinitrobenzamide phosphate ester and 2-[2-bromoethyl)-2,4-dinitro-3-({[3-(phosphonooxy)propyl]amino}carbonyl)anilino]ethyl methanesulfonate.

18. A kit comprising a compound of Formula (I) as defined in claim 1 or a pharmaceutically acceptable salt thereof, and docetaxel.

19. The kit of claim 18 in which the compound of Formula (I) is 2[(2-bromoethyl)-2,4-dinitro-6-[[[2-(phosphonooxy)ethyl]amino]-carbonyl]anilino]ethyl methanesulfonate.

20. The kit of claim 19 in which the compound of Formula (I) is selected from 2-[Bis(2-bromoethyl)amino]-N-(2-hydroxyethyl)-3,5-dinitrobenzamide phosphate ester and 2-[2-bromoethyl)-2,4-dinitro-3-({[3-(phosphonooxy)propyl]amino}carbonyl)anilino]ethyl methanesulfonate.

21. A kit comprising:
a) a compound of Formula (I) as defined in claim 1 or a pharmaceutically acceptable salt thereof in a first unit dosage form;
b) docetaxel in a second unit dosage form; and
c) container means for containing said first and second dosage forms.

22. The kit of claim 21 in which the compound of Formula (I) is 2[(2-bromoethyl)-2,4-dinitro-6-[[[2-(phosphonooxy)ethyl]amino]-carbonyl]anilino]ethyl methanesulfonate.

23. The kit of claim 21 in which the compound of Formula (I) is selected from 2-[Bis(2-bromoethyl)amino]-N-(2-hydroxyethyl)-3,5-dinitrobenzamide phosphate ester and 2-[2-bromoethyl)-2,4-dinitro-3-({[3-(phosphonooxy)propyl]amino}carbonyl)anilino]ethyl methanesulfonate.
